(19) United States
(12) Patent Application Publication
(10) Pub. No.: US 2010/0305409 A1
(43) Pub. Date: Dec. 2, 2010

(54) Tongue depressor and throat viewing mechanism

(76) Inventor: Hui-Yu Chang, Taipei (TW)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/802,049

(22) Filed: May 27, 2010

(30) Foreign Application Priority Data
Jun. 2, 2009 (TW) 098118275

(51) Int. Cl. A61B 1/24

(52) U.S. Cl. 600/240

(57) ABSTRACT

A tongue depressor and throat viewing mechanism adapted to facilitate inspection of the larynx and a medical diagnosis of its condition, the mechanism includes a blade insertable into the mouth to depress the tongue so as to expose the larynx. A camera and a light illuminating unit are located in the front end of the blade. The camera is configured to send image signals to a transmitter which is located in the rear end of the blade. The transmitter sends the images to a display for viewing.

This patent application is based on and claims priority to Taiwanese Patent Application No. 098118275, filed Jun. 2, 2009.

### BACKGROUND OF THE INVENTION

1. Field of Invention

The invention relates to tongue depressors and more particularly to a tongue depressor and throat viewing mechanism for inserting into the tongue of a patient and taking a digital video image of the larynx in the throat for further examination.

2. Description of Related Art

Medical devices for examining the mouth including the larynx are well known. For example, a tongue depressor having a flat blade is used to depress the tongue of a patient in order to fully expose the larynx so that it can be inspected by a physician. In some tongue depressor, a camera is provided behind or in the rear end of the flat blade. The camera may be attached to the tongue depressor by means of an adapter or a bracket. It is a natural response to bite the tongue depressor when the tongue depressor is placed in the open mouth of a′ patient (e.g., a baby or a child). As such, the tongue may block the view of the camera and render the camera not useful for its intended purposes. The physician may take a prolonged period of time to yield of an image of the larynx. Furthermore, it may not be possible to yield a complete image of the larynx by the rear camera. Additionally, the typical lens is too large to dispose at a front end of the tongue depressor. This can compromise the desired function of inspecting the larynx for diagnosis.

It is thus advantageous to provide a tongue depressor that is easier to use.

### SUMMARY OF THE INVENTION

It is therefore one object of the invention to provide a tongue depressor and throat viewing mechanism having a forward camera which is proximate to the larynx after inserting a blade into the mouth so that the camera may yield a clear digital image of the larynx. As such, even the tongue blocks the blade, there is no disruption or view blocking of the normal operation of the camera.

To achieve above and other objects of the invention, there is provided a tongue depressor and throat viewing mechanism adapted to facilitate inspection of the larynx of a subject and a medical diagnosis of its condition. The tongue depressor and throat viewing mechanism comprises a flat blade insertable into the mouth of the subject to depress the tongue so as to cause the throat to expose the larynx; a power source disposed on the blade; a sleeve disposed on the blade; an elongated insert comprising a rear enlargement and being adapted to insert into the sleeve to be frictionally retained by the sleeve when the enlargement is stopped at a rear end of the sleeve; a miniature, digital video camera disposed in a front end of the insert; a light emitting unit disposed in the front end of the insert; a transmitter disposed in the blade; and a switch disposed on the blade and being proximate to the power source, the switch being electrically connecting the power source to each of the camera, the transmitter, and the light emitting unit, wherein in response to inserting the blade into the mouth, activating the light emitting unit to project a light beam to illuminate the exposed larynx, activating the transmitter, and activating the camera by manually activating the switch, an image of the larynx is yielded by the camera and the image can be sent by the transmitter for exhibit.

The above and other objects, features and advantages of the invention will become apparent from the following detailed description taken with the accompanying drawings.

### BRIEF DESCRIPTION OF THE DRAWINGS

FIG. 1 is a perspective view of a tongue depressor and throat viewing mechanism according to a first preferred embodiment of the invention;

FIG. 1A is a greatly enlarged view of a front end of FIG. 1;

FIG. 2 is a cross-sectional view of FIG. 1;

FIG. 3 is a perspective view of a tongue depressor and throat viewing mechanism according to a second preferred embodiment of the invention;

FIG. 4 is a perspective view of a tongue depressor and throat viewing mechanism according to a third preferred embodiment of the invention;

FIG. 4A is a greatly enlarged view of a front end of FIG. 4;

FIG. 5 is a cross-sectional view of FIG. 4;

FIG. 6 is a perspective view of a tongue depressor and throat viewing mechanism according to a fourth preferred embodiment of the invention;

FIG. 7 is an exploded view of a tongue depressor and throat viewing mechanism according to a fifth preferred embodiment of the invention;

FIG. 8 is a block diagram of the tongue depressor and throat viewing mechanism, a channel indicator, and a computer system in cooperation therewith;

FIG. 9 is a perspective view of a tongue depressor and throat viewing mechanism according to a sixth preferred embodiment of the invention; and

FIG. 10 is a perspective view of a tongue depressor and throat viewing mechanism according to a seventh preferred embodiment of the invention.

### DETAILED DESCRIPTION OF THE INVENTION

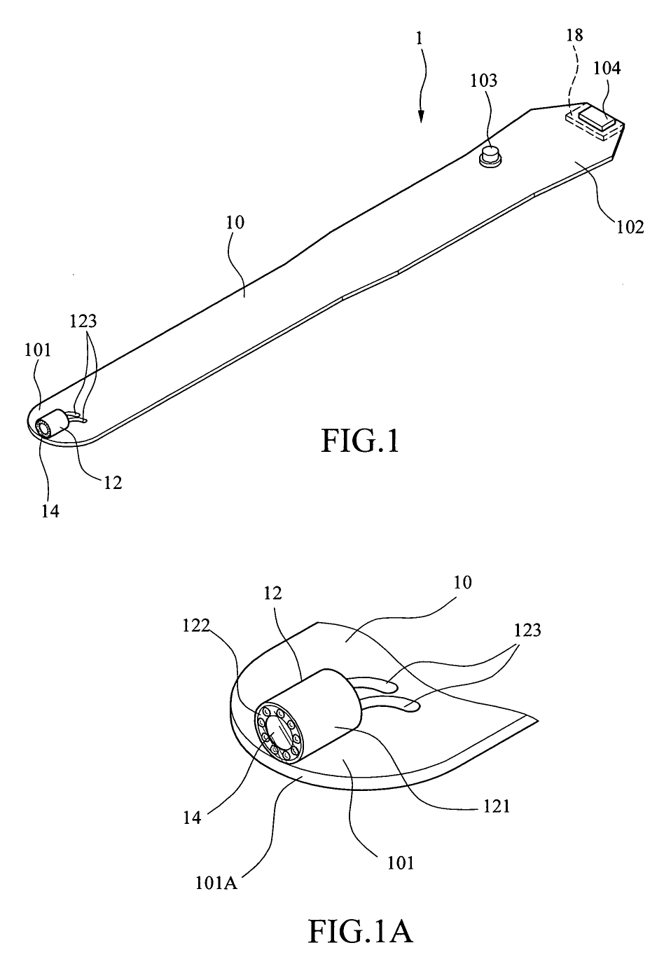

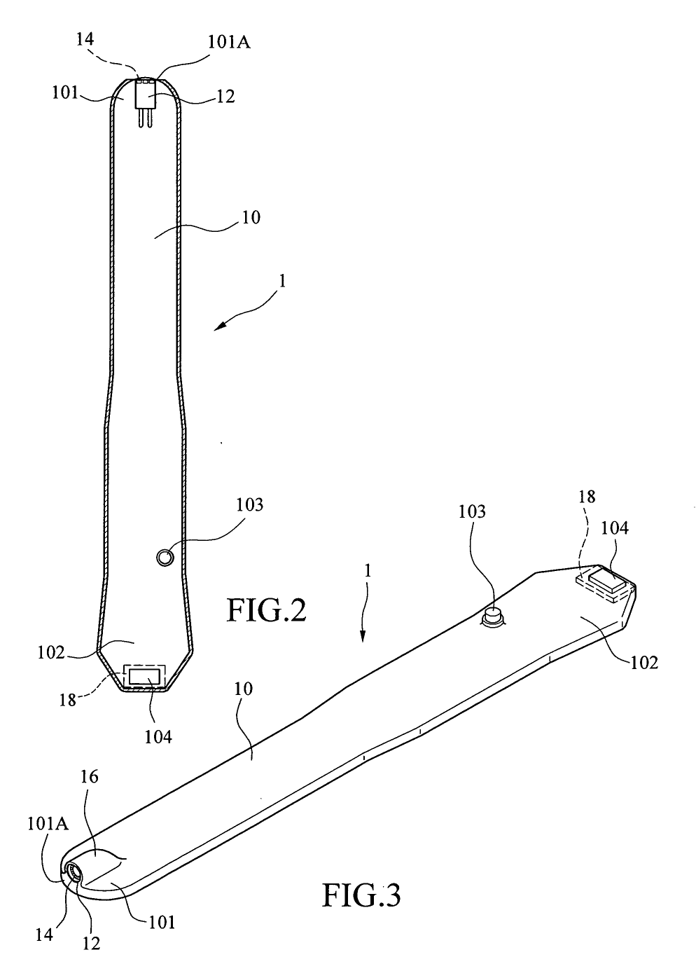

Referring to FIGS. 1, 1A and 2, a tongue depressor and throat viewing mechanism 1 in accordance with a first preferred embodiment of the invention comprises the following components as discussed in detail below.

A flat blade 10 has a front end 101 and a rear end 102. A miniature, digital video camera 12 is provided in a cylinder 121 which is mounted on top of the front end 101. A lens 122 is provided on an opening of the cylinder 121 above a curved edge 101A on the front end 101. A light emitting unit 14 is provided in the camera 12. Power and cable 123 are interconnected to the camera 12 and a printed circuit board (PCB) (not shown) is within the blade 10. A power source (e.g., battery) 104 is provided on a top position proximate to the rear end 102. A switch (e.g., push button switch) 103 is provided between the power source 104 and a center of the blade 10. The switch 103 is electrically interconnected to the power source 104 and the PCB for permitting a power supply from the power source 104 to the PCB or not. Moreover, power can be supplied from the PCB to the camera 12 and the light emitting unit 14. Preferably, the light emitting unit 14 comprises a plurality of light-emitting diodes (LEDs) 14.

In use, a physician may press the switch 103 to activate both the camera 12 and the light emitting unit 14 which thus can project a light beam. Next, insert the blade 10 into the mouth of a patient (e.g., baby or child) to press down the tongue for inducing a gag reflex. Naturally, the throat may dilate to fully expose the larynx for viewing. The camera 12, provided on the front end 101 of the blade 10, is proximate to the larynx. Thus, the camera 12 may yield a clear digital image of the larynx which is being illuminated by the light emitting unit 14 since, even the tongue or the lips of the patient block the blade 10, there would be no disruption or view blocking of the normal operation of the camera 12. The image of the larynx is then wirelessly sent by a transmitter 18 to a display (not shown) of a computer (not shown) in a magnified scale. Note that details of the wireless transmission will be discussed later. Therefore, the physician may clearly see the larynx from the display and a correct diagnosis can be made. The camera has a diameter about 5 mm, but it can be larger or smaller.

Referring to FIG. 3, a tongue depressor and throat viewing mechanism in accordance with a second preferred embodiment of the invention is characterized below. The cylinder 16 is integrally formed with the top of the front end 101. Both the camera 12 and the light emitting unit 14 are provided in the cylinder 16.

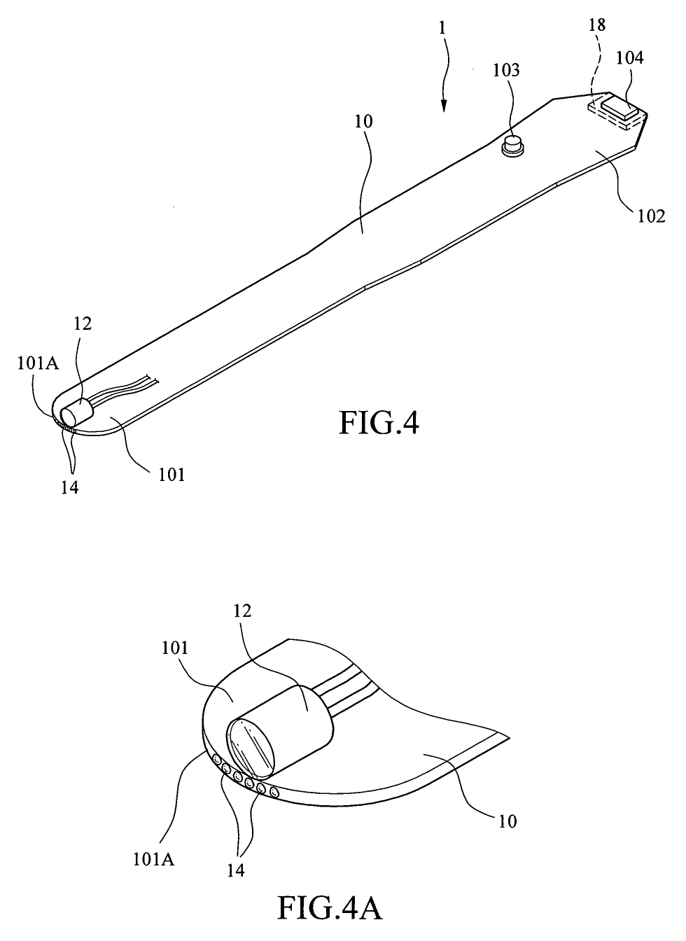

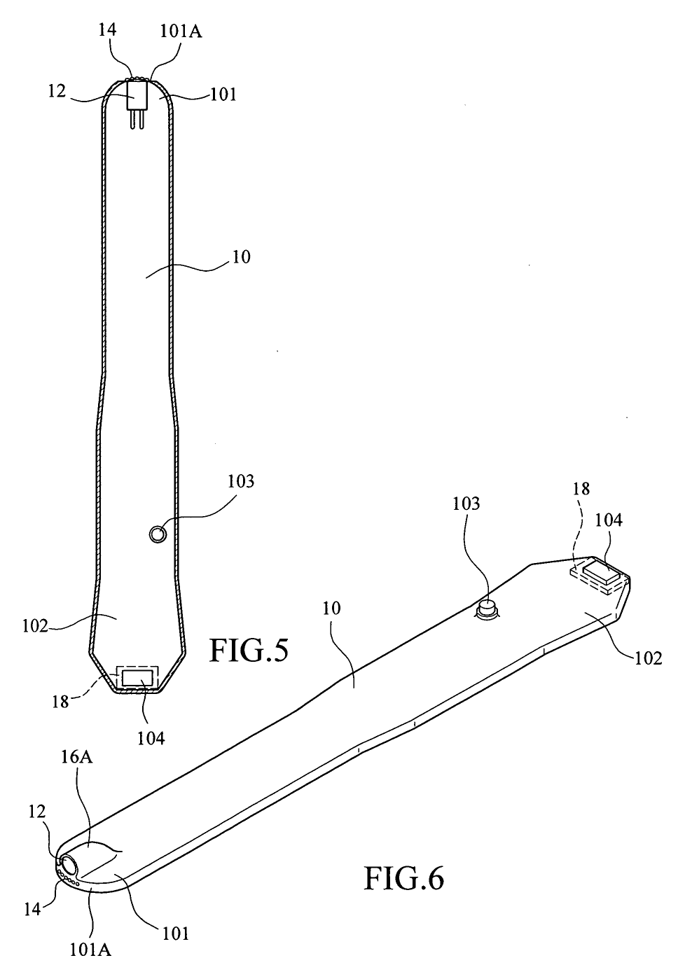

Referring to FIGS. 4, 4A and 5, a tongue depressor and throat viewing mechanism in accordance with a third preferred embodiment of the invention is characterized below. The light emitting unit 14 comprises a plurality of LEDs 14 which are provided on the curved edge 101A on the front end 101.

Referring to FIG. 6, a tongue depressor and throat viewing mechanism in accordance with a fourth preferred embodiment of the invention is characterized below. The cylinder 16A is integrally formed with the top of the front end 101. The camera 12 is provided in the cylinder 16A. The light emitting unit 14 comprises a plurality of LEDs 14 which are provided on the curved edge 101A on the front end 101.

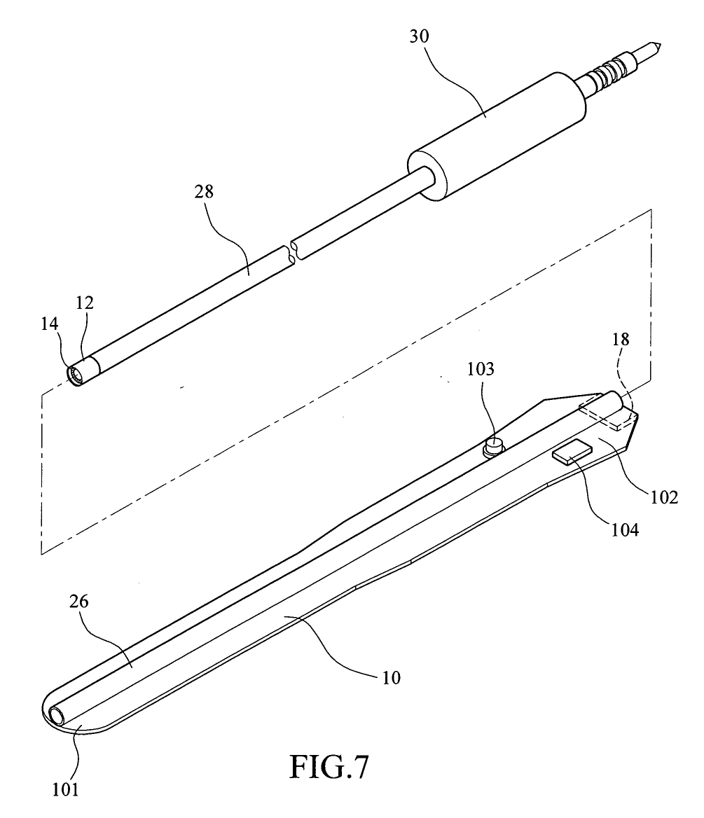

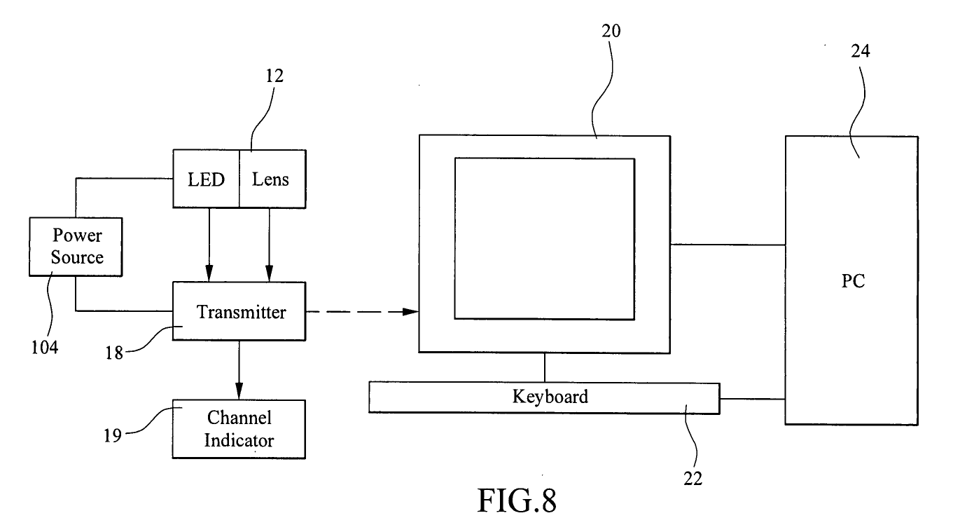

The cylinder 16A is integrally formed with the top of the front end 101. The camera 12 is provided in the cylinder 16A. The light emitting unit 14 comprises a plurality of LEDs 14 which are provided on the curved edge 101A on the front end 101. A transmitter 18 is provided on top of the rear end 102. The transmitter 18 is electrically connected to the power source 104 and is in signal communication with the camera 12. Referring to FIGS. 7 and 8, a tongue depressor and throat viewing mechanism in accordance with a fifth preferred embodiment of the invention is characterized below. A lengthwise sleeve 26 of circular section is provided on top of the blade 10. A cylindrical insert 28 has a front end provided with a camera 12 and a light emitting unit 14 and a rear end provided with an enlargement 30. The insert 28 can be inserted into the sleeve 26 until the enlargement 30 contacts the sleeve 26. At this position, the insert 28 is frictionally retained in the sleeve 26 and the camera 12 is disposed at the front end of the sleeve 26 (i.e., on top of the front end 101). Switch 103, transmitter 18, and power source 104 are provided proximate to the rear end 102. This embodiment facilitates a replacement of the camera 12 if such need arises.

As shown in FIG. 8, image signals can be sent from the camera 12 to the transmitter 18 which, in turn, wirelessly transmits the image signals to a personal computer (PC) 24 or the like. The transmitter 18 may also transmit the image signals to a channel indicator 19 by wire or by a wireless means (such as IR or Bluetooth signals). The channel indicator 19 is provided proximate to where the physician performs the examination so that the physician may see the operating channel of the transmitter 18. Moreover, the PC 24 is equipped with a display 20 for showing the magnified examination image of the larynx, and a keyboard 22.

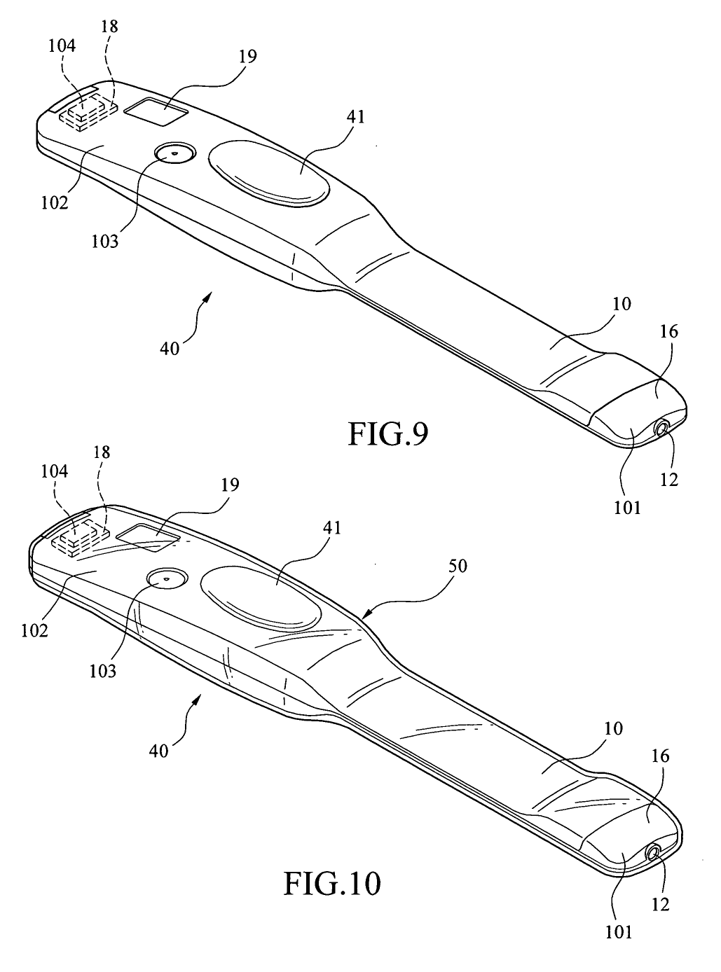

Referring to FIG. 9, a tongue depressor and throat viewing mechanism in accordance with a sixth preferred embodiment of the invention is characterized below. The blade 10 is wrapped around by a flexible sheath 40 having an oval depression 41 for ease of manual pushing of the blade 10 into the tongue. Also, the switch 103 and the channel indicator 19 are provided proximate to the rear end 102. The camera 12 is provided in the cylinder 16 which is integrally formed on top of the front end 101.

Referring to FIG. 10, a tongue depressor and throat viewing mechanism in accordance with a seventh preferred embodiment of the invention is characterized below. An additional transparent, flexible, disposable enclosure 50 made of plastic is put on the sheath 40. After a single use, the enclosure 50 can be discarded for hygienic purposes.

The invention has the following advantages: In use, after inserting the blade into the mouth of a patient to press down the tongue, the throat may dilate to fully expose the larynx for viewing. The forwardly located camera is relatively small (e.g., about or less than 5 mm in diameter) so as to enable it to dispose on a front end of the tongue depressor in the throat proximate to the larynx. As such, the camera can yield a clear digital image of the larynx. Even when the tongue or other part of the mouth blocks the blade, there is no disruption or view blocking of the normal operation of the camera. Moreover, the image of the larynx can be sent to a display for showing in a magnified scale by wireless transmission. Thus, a correct diagnosis can be made.

In summary, the present invention provides a tongue depressor and throat viewing apparatus adapted to facilitate inspection of larynx of a subject, such as a person. The apparatus has a blade (10) with a front end (101) and a rear end (102). Thee blade is configured to be inserted into the mouth of the subject so that at least the front end depresses the subject's tongue for exposing the larynx. At the front end, a camera (12) is used to take images of the larynx and it surrounding area. A light emitting unit located in relationship to the camera is used to provide lighting to the camera. The camera can send images to a transmitter (18) which is configured for transmitting the image signals for display. Transmitter can be located in the rear end of the blade, but it can be a separate unit. The apparatus also has a switch for electrically connecting the light emitting unit and the camera to a power source. The switch is manually operated when needed. The power source can be a battery of a small size so it can be attached to the rear end of the blade.

The camera can be mounted on a mount located in the front end. The light emitting unit can have one or more light emitting diodes located on a front-curved edge of the blade. Alternatively, the light emitting unit has a plurality of light emitting diodes mounted around the circumference of the camera lens. In one embodiment of the present invention, a flexible sheath (40) is provided around the blade to facilitate pushing of the blade into the mouth. In another embodiment of the present invention, a disposable wrap is for wrapping around the blade, with or without the flexible sheath. The disposable wrap (50) can be made of transparent and flexible material. In yet another embodiment, an elongated insert (28) having a first end is used for mounting the camera, and a lengthwise sleeve (26) is located on the blade, wherein the elongated insert is dimensioned for inserting through the sleeve so that the camera can be located in the front end of the blade. Preferably, the elongated insert (28) has an opposing second end with an enlarged portion for preventing the elongated insert from further inserting into the sleeve. According to the present invention, the transmitter is configured to provide an electrical signal to a channel indicator (19) for indicating an operating channel of the transmitter.

Thus, while the invention has been described in terms of preferred embodiments, those skilled in the art will recognize that the invention can be practiced with modifications within the scope of the appended claims.

What is claimed is:

1. A tongue depressor and throat viewing apparatus adapted to facilitate inspection of larynx of a subject, said apparatus comprising:
a blade (10) having a front end (101) and a rear end (102), the blade configured to insert into the mouth of the subject so that at least the front end depresses the subject's tongue for exposing the larynx;
a camera (12) located in the front end; and
a light emitting unit (14) located in relationship to the camera for providing lighting to the camera, wherein the camera is configured to provide image signals indicative of images captured by the camera.

2. An apparatus according to claim 1, further comprising:
a transmitter (18) configured for receiving the image signals and for transmitting the image signals for display.

3. An apparatus according to claim 2, wherein the transmitter is located in the rear end of the blade (10).

4. An apparatus according to claim 1, further comprising:
a switch (103) for electrically connecting the light emitting unit (14) and the camera (12) to a power source.

5. An apparatus according to claim 2, further comprising:
a power source (104) located near the rear end (102); and
a switch (103) located near the rear end configured for electrically connecting the light emitting unit (14) and the camera to the power source.

6. An apparatus according to claim 5, wherein the switch is also configured for electrically connecting the transmitter to the power source.

7. An apparatus according to claim 1, further comprising:
a mount disposed in the front end for mounting the camera and the light illuminating unit.

8. An apparatus according to claim 1, wherein the front end of the blade comprises a curved edge (101A), and the light emitting unit (14) comprises one or more light-emitting diodes disposed on the curved edge.

9. An apparatus according to claim 1, wherein the camera comprises a camera lens and the light emitting unit (14) comprises a plurality of light-emitting diodes surrounding the lens.

10. An apparatus according to claim 1, further comprising:
a flexible sheath (40) around the blade for facilitating pushing of the blade into the mouth.

11. An apparatus according to claim 1, further comprising:
a disposable wrap (50) for wrapping around the blade.

12. An apparatus according to claim 1, further comprising:
an elongated insert (28) having a first end for mounting the camera; and
a lengthwise sleeve (26) disposed on the blade, wherein the elongated insert is dimensioned for inserting through the sleeve so that the camera can be located in the front end of the blade.

13. An apparatus according to claim 12, wherein the elongated insert (28) has an opposing second end with an enlarged portion for preventing the elongated insert from further inserting into the sleeve (26).

14. An apparatus according to claim 2, wherein the transmitter is configured to provide an electrical signal to a channel indicator (19) for indicating an operating channel of the transmitter.

15. An apparatus according to claim 1, wherein the camera has a diameter substantially equal to or smaller than 5 mm.

16. An apparatus according to claim 11, wherein the disposable wrap (50) is made of transparent and flexible material.
